(19) United States
(12) Patent Application Publication
(10) Pub. No.: US 2025/0157676 A1
(43) Pub. Date: May 15, 2025

(54) SYSTEMS AND METHODS FOR DETERMINING ELECTRONIC DATA ASSOCIATIONS BETWEEN A SUBJECT AND A CONDITION

(71) Applicant: Jona, Inc., Stamford, CT (US)

(72) Inventors: Leo GRADY, Stamford, CT (US); Jonas SCHLUETER, Stamford, CT (US); Clay H. YOO, Stamford, CT (US); Kfir BAR, Tel Aviv (IL)

(73) Assignee: Jona, Inc., Stamford, CT (US)

(21) Appl. No.: 18/947,885

(22) Filed: Nov. 14, 2024

Related U.S. Application Data
(60) Provisional application No. 63/598,804, filed on Nov. 14, 2023.

(51) Int. Cl. G16H 70/60; G16H 50/70

(52) U.S. Cl. CPC G16H 70/60; G16H 50/70

(57) ABSTRACT

Systems and methods for determining an association between a subject and at least one condition are disclosed. One computer-implemented method may include: receiving population biomarker data from a plurality of systems that include study data for a plurality of studies; receiving one or more population probability values; receiving one or more biomarker levels associated with the subject; analyzing, using a processor associated with the system, the one or more biomarker levels against the population biomarker data and the one or more population probability values; generating, based on the analyzing, an association score between the one or more biomarker levels and the at least one condition; and outputting the association score to a display of a computing device.

### CROSS-REFERENCE TO RELATED APPLICATION(S)

This application claims priority to U.S. Provisional Application No. 63/598,804, filed Nov. 14, 2023, which is incorporated by reference herein in its entirety.

### TECHNICAL FIELD

The present disclosure relates generally to the field of computational analysis of biological data and, more specifically, to systems and methods for aggregating and applying biomarker study data to interpret new biomarker data in relation to conditions, populations, and potential interventions.

### BACKGROUND

Biomarker research has rapidly expanded, producing a wealth of data linking various biomarkers to diseases, health conditions, and treatment responses. However, studies regarding similar biomarkers often diverge in terms of subject populations, methodologies, and results, creating challenges in synthesizing these disparate studies for consistent and actionable insights. Modern artificial intelligence (AI) technologies offer the capability to ingest large amounts of scientific literature, but the challenge remains in integrating findings from different studies to create reliable interpretations of new data.

The present disclosure is accordingly directed to techniques for combining biomarker studies and utilizing the combined data to analyze new biomarker samples and provide potential actions. The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for combining and analyzing biomarker studies to determine association scores between biomarker levels in a subject and specific conditions or responses.

In one aspect, a computer-implemented for determining an association between a subject and at least one condition is provided. The computer-implemented method may include operations including: receiving, at a computing device associated with a system, population biomarker data from a plurality of systems that include study data for a plurality of studies; receiving, at the computing device, one or more population probability values; receiving, at the computing device, one or more biomarker levels associated with the subject; analyzing, using a processor associated with the system, the one or more biomarker levels against the population biomarker data and the one or more population probability values; generating, based on the analyzing and using the processor, an association score between the one or more biomarker levels and the at least one condition; and outputting, using the processor, the association score to a display of the computing device.

In another aspect, a system for determining an association between a subject and at least one condition is provided. The system may include: one or more processors; and one or more computer readable media storing instructions that are executable by the one or more processors to perform operations comprising: receiving population biomarker data from a plurality of systems that include study data for a plurality of studies; receiving one or more population probability values; receiving one or more biomarker levels associated with the subject; analyzing the one or more biomarker levels against the population biomarker data and the one or more population probability values; generating, based on the analyzing and using the processor, an association score between the one or more biomarker levels and the at least one condition; and outputting, using the processor, the association score to a display of the computing device.

In yet another aspect, a non-transitory computer-readable medium storing computer-executable instructions is provided. The computer-executable instructions, when executed by a server in network communication with at least one database, cause the server to perform operations including: receiving, at a computing device associated with a system, population biomarker data from a plurality of systems that include study data for a plurality of studies; receiving, at the computing device, one or more population probability values; receiving, at the computing device, one or more biomarker levels associated with the subject; analyzing, using a processor associated with the system, the one or more biomarker levels against the population biomarker data and the one or more population probability values; generating, based on the analyzing and using the processor, an association score between the one or more biomarker levels and the at least one condition; and outputting, using the processor, the association score to a display of the computing device.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1 depicts a block diagram of an exemplary computer system, according to one or more aspects of the present disclosure.

FIG. 2 depicts an exemplary workflow for identifying an association between biomarkers of a subject and a condition, according to one or more embodiments of the present disclosure.

FIG. 3 depicts an example computing system, according to one or more embodiments of the present disclosure.

### DETAILED DESCRIPTION OF EMBODIMENTS

The rapid advancement of biomarker research has created a rich but complex body of knowledge linking biological measurements to various health conditions, prognoses, and treatment responses. However, synthesizing and utilizing this wealth of information poses significant challenges. More particularly, issues reside in the sheer volume and heterogeneity of biomarker studies, which may differ in terms of subject populations, research methodologies, data collection protocols, and analytical techniques. This variability makes it difficult to merge findings across studies into a cohesive framework that may be used to interpret new data effectively and provide actionable insights. Moreover, the pace of the scientific publication process means that any comprehensive review may quickly become outdated, thereby limiting the practical utility of conventional methods for real-time data analysis.

Conventional attempts to solve this problem have primarily included manual meta-analyses and systematic reviews, which may aggregate data from multiple studies to identify common findings. These methods, while valuable for understanding broader trends in the literature, are limited in their scalability. The manual nature of meta-analyses means they are time-consuming, prone to human error, and restricted by the number of studies that may be feasibly reviewed. Systematic reviews face similar limitations and often provide only qualitative or semi-quantitative insights, without yielding dynamic, personalized interpretations of individual biomarker data. Basic computational tools, while offering some level of data aggregation, often fall short in addressing the complex task of reconciling disparate study methodologies and outcomes. Specifically, these tools typically cannot standardize diverse study data effectively or adjust their analyses to reflect the varying quality and reliability of different studies.

The limitations of conventional methods create several issues. First, manual and semi-automated approaches may not keep pace with the increasing volume of scientific literature, leading to incomplete or outdated analyses. Second, existing computational methods often lack the sophistication needed to standardize findings from studies that employ different research designs or feature diverse subject demographics, leading to biased or incomplete results. Third, conventional approaches rarely incorporate real-time updates or dynamic learning, making them inflexible for applications that require continuous integration of new data. Finally, conventional tools and analyses are typically one-dimensional, failing to account for differences in study quality or robustness, which may skew results if low-quality studies disproportionately influence the analysis.

The present disclosure offers a solution to these challenges by introducing a system and method for combining biomarker studies in a comprehensive, automated, and adaptable manner. The concepts described herein may leverage modern AI technologies, such as natural language processing (NLP) and machine learning, to ingest, normalize, and synthesize a wide range of biomarker studies. In an aspect, the system may utilize quality scores assigned to each study based on criteria such as sample size, journal impact factor, and citation count. These scores may help ensure that higher-quality studies contribute more significantly to the overall analysis. The system may also integrate probability distributions from various studies to form baseline models that may be used to interpret new biomarker data from individual subjects.

In an aspect, the novel process may involve calculating association scores that link the biomarker levels of a subject to specific conditions or populations. These scores may be determined through quality-weighted analyses of multiple studies, providing a nuanced and reliable interpretation of the subject's biomarker profile. Additionally or alternatively, the system may support real-time integration of new data, allowing it to remain current and relevant as new findings are published. This dynamic capability may address the limitations of static, conventional methods and ensure that users can access the most up-to-date interpretations and recommendations.

The concept summarized above, and further elaborated upon herein, addresses the limitations of conventional approaches by offering a scalable, automated, and adaptive solution that synthesizes findings from disparate biomarker studies into coherent, actionable insights. By integrating quality-weighted analysis, real-time updates, and machine learning-based refinement, the concepts overcome the issues of scalability, standardization, outdated information, and limited analytical depth. This enables the system to provide personalized health assessments, actionable recommendations, and robust interpretations of biomarker data, fulfilling a need in the field of biomarker research and analysis.

The novel concepts improve computer technology and the technical field of biomarker analysis by leveraging advanced AI-driven methods to enhance the synthesis and interpretation of complex biological data. Traditional systems struggle to process and combine the rapidly growing and heterogeneous body of biomarker studies effectively. The novel system addresses these limitations by integrating NLP and machine learning techniques to automate the ingestion and normalization of diverse scientific literature. By doing so, the system optimizes computational resources, enabling the analysis of vast amounts of data at a scale and speed unattainable by conventional methods. Moreover, the system introduces a novel approach to weighting study data based on quality metrics, ensuring that high-confidence data exerts greater influence on outcomes. This capability enhances the reliability and accuracy of outputs compared to earlier, more static computational tools. The dynamic, real-time updating feature allows the system to remain current as new studies are published, overcoming the static nature of traditional reviews meta-analyses that quickly become outdated. By automating the synthesis of disparate data sources into actionable insights, the concepts described herein reduce the burden on human analysts and mitigates the risk of human error, significantly advancing the field's ability to process complex biological information.

Additionally to the foregoing, the system's use of machine learning to refine association scores represents a substantial technical advancement. This feature enables iterative learning from historical data to improve the accuracy of predictive analyses, making the system adaptive and more precise over time. These enhancements to computational processing not only facilitate more comprehensive and real-time interpretations but also extend the practical applications of biomarker research to personalized medicine, diagnostics, and treatment recommendations. By improving the efficiency, accuracy, and scalability of how biomarker data is analyzed and applied, the concepts described herein make a meaningful contribution to both computer technology and the broader technical field of bioinformatics.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

The terminology used may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of +10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “electronic application” or “application” may be used interchangeably with other terms like “program,” or the like, and generally encompasses software that is configured to interact with, modify, override, supplement, or operate in conjunction with other software.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

As used herein, a “biomarker” may refer to one or more measurements of one or more biological samples.

As used herein, a “biological sample” may include, but is not limited to, whole organisms, parts of organisms, in vivo, in vitro (e.g., ex vivo), etc. The biological sample may be taken from a population (e.g., wastewater sampling) or from the environment interacting with a biological entity (e.g., air quality). The organism may be living or dead, human or nonhuman, etc. Examples of measurements may include, but are not limited to, blood components, urine components, stool components, skin samples, genetics, genomics, transcriptomics, metabolomics, microbiome data, proteomics, wearable data, home health data, environmental factors, tissue samples, etc. In some aspects, there are multiple possible values of the biomarker that may be ordered (e.g., cholesterol levels, heart rate, relative abundance of a microbe species, etc.). For clarity, the biomarker values may be continuous valued (e.g., cholesterol level), discrete valued (e.g., a Gleason score) or discretized continuous valued.

As used herein, a “microbiome” may refer to the composition of one or more portions of the entire aggregate of all microbiotas (e.g., including all related microbiota biological properties such as genetics, proteomes, metabolomes, transcriptomomes, etc.) and properties of the environment that they reside on and/or within tissues and biofluids along with the corresponding anatomical sites in which they reside, including the skin, mammary glands, seminal fluid, uterus, placenta, ovarian follicles, lung, saliva, oral mucosa, nasal mucosa, conjunctiva, biliary tract, and gastrointestinal (GI) tract. For clarity, note that the anatomical site may be represented at one or more levels of specificity (“GI tract”, “duodenum”, “upper duodenum”, etc.). Note that the anatomical sites may vary from subject to subject (e.g., for plant versus animal) and that the subject may be living or dead (necrobiome). Types of microbiotas in our definition may include bacteria, archaea, fungi, protists, viruses, phages, plasmids, prions, parasites, mobile genetic elements and micro-animals.

As used herein, “microbiome abundance”, may refer to a set of data describing the sampled microbiome that describes the magnitude of detection within the sample for one or more elements of the microbiome analysis type (e.g., how many of different species were found, the abundance of different metabolic pathways that were detected, the abundance of different antimicrobial resistance factors that were detected, etc.). Microbiome abundance data may or may not be normalized (e.g., to form a relative abundance) or absolute (e.g., from a weighed or counted abundance). Additionally, microbiome abundance data may represent a subset of the data collected from the sample (e.g., a taxonomic analysis type may be subset to examine only the bacteria kingdom, the fungal kingdom, etc.).

As used herein, a “user” may refer to a person or machine who is interacting with the outputs. Note that the user may or may not be the same individual as the subject. Additionally, or alternatively, the term “user” may generally encompass any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.).

As used herein, a “study” may refer to a scientific study comparing one or more biomarkers between two populations of samples (e.g., a healthy vs diseased population). Studies may be published in peer-reviewed journals or may be published in a less formal venue (e.g., a preprint, blog post, press release, anecdotal, case report, etc.). In the context of this application, the terms “study” and “finding” are utilized interchangeably.

As used herein, an “action” may refer to one or more changes to the subject. Some examples of actions may include, but are not limited to, dietary changes, dietary supplements, physical activity, sleep, mental exercises (e.g., meditation), environmental changes, lifestyle changes, drugs or medications, proximity to other organisms, etc.

As used herein, a “condition” may refer to an attribute of the subject which may include, but is not limited to, disease, health, traits, allergies, sensitivity, behavior, yield, response to a drug, response to a diet, etc.

As used herein, a “population” may refer to any distinct group of subjects. Distinction can be made by demographics (e.g., age, gender, geographic location, ethnicity), subjects with a certain condition (e.g., diabetics, individuals with food allergy, individuals with shellfish allergy, etc.), prognosis (e.g., subjects who develop colon cancer, subjects who respond to a medication, etc.), treatment type (e.g., subjects given a vegetarian diet, subjects who received surgery, subjects who received chemotherapy, etc.)

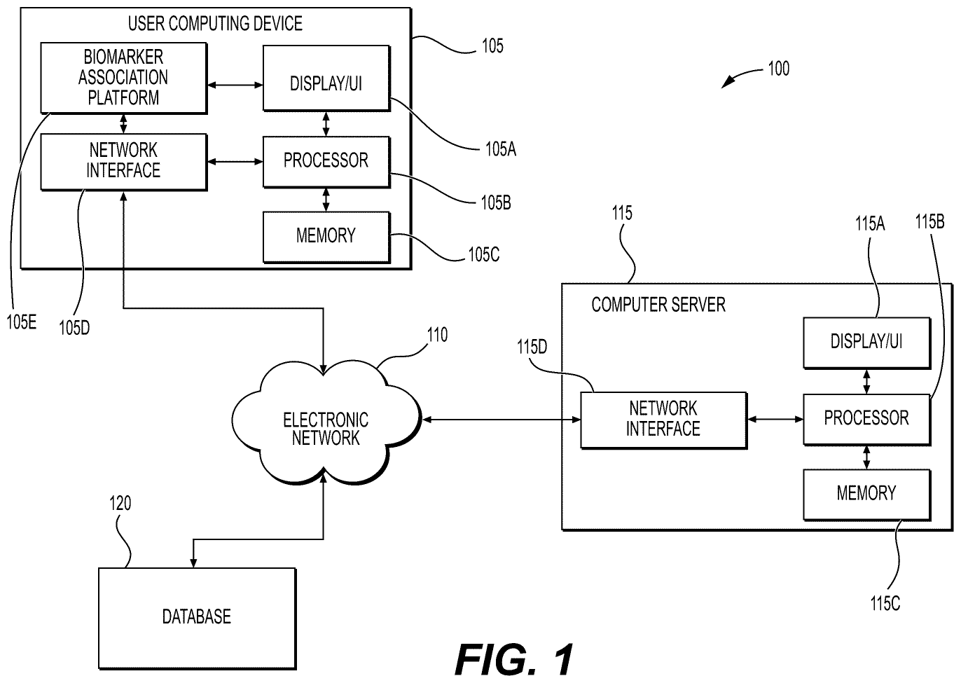

Referring now to FIG. 1, a block diagram depicting an exemplary system environment 100 for improving healthcare and, more specifically, for interpreting new biomarker data, is provided. The system environment 100 may include a computing device 105 operated by a user, an electronic network 110, a server (“computer server”) 115, and a database 120. Each of the foregoing components may be connected via the electronic network 110, e.g., using one or more standard wired, data transfer and/or wireless communication protocols, and/or other means known to those skilled in the art but not explicitly listed here. The system environment 100, such as computer server 115, may include one or more computing devices. If the one or more processors of the computer system 100 are implemented as a plurality of processors, the plurality of processors may be included in a single computing device or distributed among a plurality of computing devices. If a computer server 115 comprises a plurality of computing devices, the memory of the computer server 115 may include the respective memory of each computing device of the plurality of computing devices. The computer server 115 and the database 120 may be one server computer device and a single database, respectively. Alternatively, the computer server 115 may be a server cluster, or any other collection or network of a plurality of computer servers. The database 120 also may be a collection of a plurality of interconnected databases. The computer server 115 and the database 120 may be components of one server system 100.

Additionally, or alternatively, the computer server 115 and the database 120 may be components of different server systems, with the electronic network 110 serving as the communication channel between them.

The computing device 105 may include a display/user interface (UI) 105A, a processor 105B, a memory 105C, a network interface 105D, and/or a biomarker association application (“application”) 105E. The user computing device 105 may be a personal computer (PC), a tablet PC, a television (TV), a smart TV a personal digital assistant (PDA), a mobile device, a palmtop computer, a laptop computer, a desktop computer, etc. The user computing device 105 may execute, by the processor 105B, an operating system (O/S) and at least one application (each stored in memory 105C). The application 105E may be a browser program or a mobile application program (which may also be a browser program in a mobile O/S). Users may be able to provide inputs to and receive outputs from the application 105E via interaction with one or more digital icons resident thereon. In some embodiments, outputs provided by the application 105E may be facilitated based on instructions/information stored in the memory 105C. The output may be visual data presented on the application GUI and may be executed, for instance, based on XML and Android programming languages or Objective-C/Swift. However, one skilled in the art would recognize that this may also be accomplished by other methods, such as webpages executed based on HTML, CSS, and/or scripts, such as JavaScript. The display/UI 105A may be a touch screen or a display with other input systems (e.g., mouse, keyboard, etc.). The network interface 105D may be a TCP/IP network interface for, e.g., Ethernet or wireless communications with the network 110. The processor 105B, while executing the application 105E, may receive user inputs from the display/UI 105A, and perform actions or functions in accordance with the application or other related applications.

The computer server 115 may include a display/UI 115A, a processor 115B, a memory 115C, and/or a network interface 115D. The computer server 115 may be a computer, system of computers (e.g., rack server(s)), and/or or a cloud service computer system. The computer server 115 may execute, by the processor 115B, an operating system (O/S) and at least one instance of a server program (each stored in memory 115C). The computer server 115 may store or have access to information from the database 120. The display/UI 115A may be a touch screen or a display with other input systems (e.g., mouse, keyboard, etc.) for an operator of the computer server 115 to control the functions of the computer server 115 (e.g., update the server program and/or the server information). The network interface 115D may be a TCP/IP network interface for, e.g., Ethernet or wireless communications with the network 110.

The system environment 100 described herein may include one or more processors (e.g., processors 105B and/or 115B) that are configured to execute instructions to train and/or implement a machine learning model. As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of a machine-learning model may include deployment of one or more machine-learning techniques, such as k-nearest neighbors, linear regression, logistical regression, random forest, gradient boosted machine (GBM), support-vector machine, deep learning, a deep neural network, and/or any other suitable machine-learning technique that solves problems in the field of Natural Language Processing (NLP). Supervised, semi-supervised, and/or unsupervised training may be employed. For example, supervised learning may include providing training data and labels corresponding to the training data, e.g., as ground truth. Unsupervised approaches may include clustering, classification, or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Prior to introduction to a machine learning infrastructure, data may be processed and normalized. As used herein, the term “normalize” may refer to the transformation of a value or a set of values to a common frame of reference for comparison purposes. In this regard, one or more normalization algorithms or techniques (e.g., min-max normalization, z-score normalization, decimal scaling, logarithmic transformation, root transformation, etc.) may be leveraged to bring all data attributes in the context data onto the same scale. Such a process may correspondingly improve the performance of the machine learning model by reducing the impact of any outliers and by improving the accuracy of a trained machine learning model associated therewith.

In some embodiments, a machine-learning model based on neural networks includes a set of variables, e.g., nodes, neurons, filters, etc., that are tuned, e.g., weighted or biased, to different values via the application of training data. In other embodiments, a machine learning model may be based on architectures such as support-vector machines, decision trees, random forests or Gradient Boosting Machines (GBMs). Alternate embodiments include using techniques such as transfer learning, wherein one or more pre-trained machine learning models on large common or domain specific dataset may be leveraged for analyzing the training data.

In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn semantic associations between the raw data and the context with which it is associated with (e.g., aspects of the industrial or professional field that the raw data is associated with, etc.), such that the trained machine-learning model is configured to provide output features that are contextually relevant for a user's purpose.

In various embodiments, the variables of a machine-learning model may be interrelated in any suitable arrangement in order to generate the output. For example, in some embodiments, the machine-learning model may include signal processing architecture that is configured to identify, isolate, and/or extract features, patterns, and/or structure in a text. For example, the machine-learning model may include one or more convolutional neural network (“CNN”) configured to identify characteristics of caption data obtained from a live media stream, and may include further architecture, e.g., a connected layer, neural network, etc., configured to determine a relationship between the words and phrases to summarize the most important aspects of the captioned content.

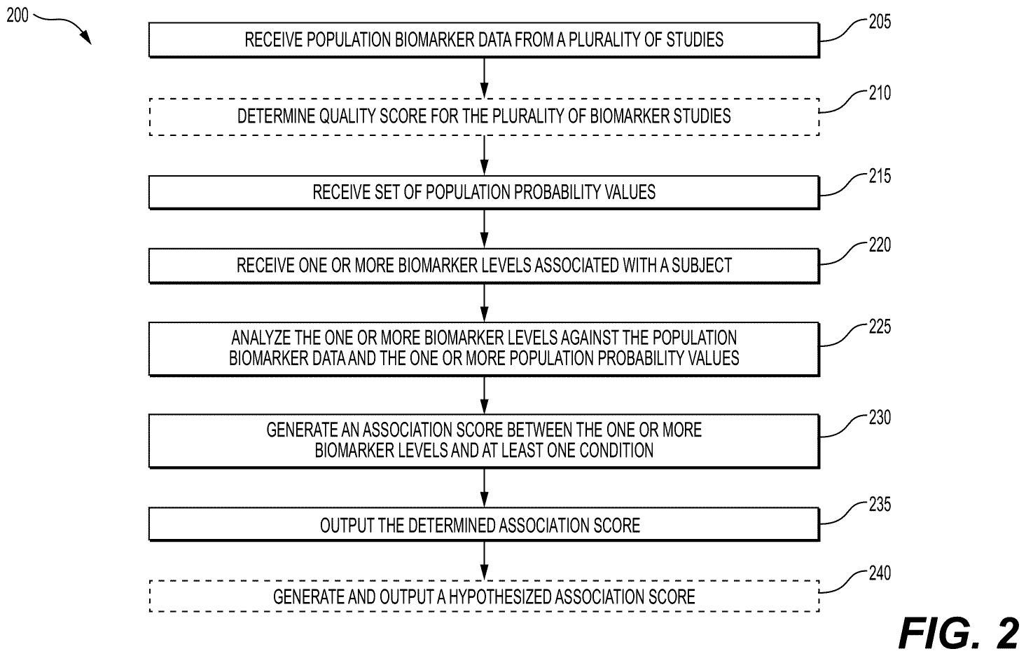

Referring now to FIG. 2, an exemplary process flow 200 for determining an association between subject biomarker data and a condition is provided, according to one or more embodiments of the present disclosure. Exemplary process flow 200 may be implemented by any combination of components included in system environment 100.

At step 205, data from a diverse range of scientific studies that analyze biomarkers in different populations may be gathered and processed. In an aspect, the system may be capable of ingesting studies that compare one or more biomarkers across various populations linked to specific conditions (e.g., a healthy population versus a diseased population, or a group of drug responders versus non-responders). These studies may originate, for example, from one or more distinct data sources or subsystems that each contain information derived from various independent scientific studies related to biomarkers. For example, the studies may include peer-reviewed journals, preprints, reports, or other reputable sources. The system may leverage advanced AI, such as one or more NLP techniques, to automatically read, interpret, and extract relevant data from these studies, which may include one or more of: the types of biomarkers studied, observed differences between populations, study design details, and reported statistical outcomes.

In an aspect, the collected studies may vary widely in terms of methodologies, analyzed populations, and reported findings. This variability may reflect real-world diversity in research conditions and subjects. By compiling these studies, the system may lay the groundwork for a robust and representative analysis that accommodates differences in subject demographics, sampling methods, and/or experimental designs. The system may also account for data structures such as comparative tables, figures, and descriptive text that provide insight into how biomarkers are associated with specific conditions. This step may ensure that the system has a rich, multifaceted data set to draw upon, enabling it to conduct a thorough and meaningful interpretation when comparing a subject's biomarker levels to those found in studied populations.

At optional step 210, a quality score may be determined for each biomarker study to weight its contribution accurately in subsequent analyses. This score may act as a measure of the study's strength and may be based on several factors. For instance, the sample size may be an important metric, as larger studies tend to offer more statistically significant and generalizable results. Additionally, the impact factor of the journal in which the study was published may serve as a proxy for the study's perceived credibility and peer-review rigor. Other contributing factors may include the number of citations the study has received (e.g., indicating its influence and acceptance in the scientific community), as well as the publication date, in order to ensure that more recent, possibly more advanced, studies are prioritized.

In an aspect, the system may combine these factors in various ways to produce a single, comprehensive quality score. This combination may involve mathematical techniques such as adding or multiplying scores for each factor, with optional normalization to ensure that scores fall within a standardized range (e.g., between 0 and 1, between 0% and 100%, etc.). Normalization may help maintain consistency across studies that may otherwise differ in how their quality metrics are quantified. By assigning quality scores, the system may effectively filter and weigh the data, allowing studies with higher scores to exert more influence on the final analysis, while studies with lower quality scores may contribute less. Additionally or alternatively, studies with lower quality scores that do not exceed a predetermined threshold may be excluded from being utilized in downstream analysis. This approach may therefore mitigate the risk of skewed results that may arise if low-quality or less reliable studies were given equal weight.

At step 215, a set of population probability values may be received that describe the distribution of biomarker levels within different populations. These values may provide the baseline against which a subject's biomarker data may be compared. In an aspect, these population probability values may be obtained from various sources. For instance, these values may be generated from raw biomarker data in large datasets that are derived from multiple studies or public biomedical databases. In some cases, the system itself may maintain or generate pre-processed population probability values based on previously ingested and analyzed studies. In an aspect, the probability values may be represented in several formats, such as histograms that show the frequency distribution of observed biomarker levels across a population, or probability density functions (PDFs) that offer a continuous representation of how biomarker values are spread. These PDFs may be constructed from histograms using techniques such as kernel density estimation, where a chosen kernel (e.g., Gaussian) may help smooth the distribution to create a clearer probability model.

Additionally or alternatively to the foregoing, in an aspect, probability distributions may be represented using mathematical models that assume a specific distribution shape (e.g., Gaussian, uniform, or binomial) based on observed data. This modeling may help approximate how biomarker levels are expected to vary within a given population, providing a structured way to compare different populations with varying characteristics. For example, a Gaussian distribution may be modeled using its mean and standard deviation, offering a straightforward way to describe the data's central tendency and variability. Collectively, these population probability values may be important for determining how the subject's biomarker levels align with those found in different populations, such as healthy individuals or those with a particular condition. By incorporating these probabilistic representations, the system may apply statistical methods to calculate how likely it is that the subject's biomarker levels match those of each population under consideration.

At step 220, one or more biomarker levels associated with a subject may be received. More particularly, the subject's biomarker levels may serve as the input data that may be evaluated against the comprehensive dataset of studies and probability distributions received in earlier steps. These biomarkers may represent a range of biological measurements, such as blood component levels, genetic markers, microbiome compositions, or other health indicators relevant to the subject's profile. In some aspects, the received biomarker levels may be standardized to ensure consistency when comparing them against the probabilistic models derived from the population data. This may involve converting raw biomarker data into formats compatible with the models, such as normalizing continuous values or categorizing discrete values according to predefined scales.

In an aspect, the subject biomarker data may be received from various sources that directly measure or collect data on the subject's biological samples. In one aspect, biomarker levels may be obtained from a clinical laboratories that perform tests on biological samples, such as blood, urine, or tissue samples. In another aspect, the biomarker levels may be received from point-of-care devices that are configured to measure biomarkers directly and may provide real-time data to the system. In yet another aspect, a subject's biomarker data may be received from electronic health records (HER) systems that have access to the subject's health record.

At step 225, the one or more biomarker levels may be analyzed against the population biomarker data and the one or more population probability values. This analytical process may involve leveraging statistical and computational methods to evaluate how closely the subject's biomarker readings align with patterns observed in different reference populations. In this analysis, the system may employ probability density functions, cumulative distribution functions, or other statistical models, depending on the biomarker type, to establish a baseline comparison. For continuous biomarkers, such as cholesterol or blood glucose levels, the system may compare the subject's readings against Gaussian or other probability distributions to evaluate whether their values fall within typical ranges for a healthy population or deviate toward ranges indicative of specific conditions. In some aspects, the system may leverage the aforementioned quality scores assigned to studies in the population dataset to weight the reliability of each reference population. High-quality studies contribute more significantly to the association scores, ensuring that the comparison prioritizes robust, well-supported findings.

At step 230, an association score representative of a relationship between a subject's biomarker levels and one or more conditions may be generated based on the analyzed data. This association score may represent the strength of the link between the biomarker levels and the target conditions. This process may be conducted in several steps and may be approached through different methods, each designed to weigh and integrate findings from diverse populations and study results effectively.

In a first approach, the system may examine each individual study to calculate the likelihood that the subject's biomarker level would appear in each study-defined population. For example, if a study compares biomarker distributions between a healthy and a diseased population, the system may calculate the likelihood that the subject's biomarkers match those found in either population, based on observed data and probability values derived from that study. In an aspect, once these likelihoods are determined for each population, the system may then calculate an association score, which may quantify the subject's relationship to a particular population. This score may be derived using various statistical methods, e.g., maximum likelihood (e.g., selecting the population that maximizes the likelihood of the subject's biomarker values), maximum a priori probability (e.g., integrating prior probabilities for each population), or a weighted average of likelihoods that factors in the quality of each study, where high-quality studies may contribute more weight to the score. In an aspect, by evaluating the subject's likelihood across multiple populations and conditions, the system may assign the association score to the condition associated with the population yielding the highest score.

In a second approach, the system may evaluate each study in terms of how strongly the subject's biomarker levels are associated with specific populations or conditions. Here, instead of calculating likelihoods, the system may directly assess the subject's biomarker levels against each study's findings to produce an association score for each population or condition. To refine the final association score, the system may combine the association scores from each study, aggregating them through statistical means such as weighted mean, median, or mode. Other techniques, like minimax, may be used to consider the range of scores, enabling a conservative estimate that limits the impact of outliers. This combined score then represents the overall association between the subject's biomarker levels and the condition(s), providing a robust measure that draws on the cumulative data from multiple studies.

At step 235, the system may output the determined association scores to the user and/or a designated storage location. This step ensures that the calculated insights may be made accessible in a clear and actionable format, facilitating the insight's use for diagnostic, therapeutic, or research purposes. The results, which reflect the association between the subject's biomarker levels and specific conditions or populations, may be displayed on various platforms, such as a user interface on a computer, a mobile device, or a network-connected dashboard. In an aspect, the output may include the association score itself along with contextual information to aid interpretation. In addition to direct display, the system may store these results electronically in formats compatible with hard drives, network drives, decentralized storage, or cloud servers.

At step 240, an optional extension of the primary analysis may occur to provide deeper insights into how potential interventions may affect the subject's association with specific conditions. In an aspect, this step may begin by receiving proposed actions that may influence biomarker levels, such as dietary changes medication, or lifestyle adjustments. The system may then incorporate data from studies that compare populations that have undergone the proposed actions to those that have not. This comparative analysis may help determine whether and how these actions lead to significant changes in biomarker levels. The system may then simulate the impact of the proposed actions by adjusting the subject's biomarker data according to observed changes reported in these studies. For example, if a study shows that a certain medication results in higher or lower biomarker values for a specific group, the system may modify the subject's biomarker profile to create a “hypothesized” profile reflecting the potential effects of the action. The association score is then recalculated based on this new, hypothesized profile, following the same methodology as in the primary analysis. This recalculated score-known as the hypothesized association score-offers a projection of how the action may shift the subject's risk profile or condition association. In an aspect, the difference between the original association score and the hypothesized score may quantify the potential change resulting from the action, thereby providing actionable insights into whether the proposed intervention may be beneficial or detrimental. The results of this evaluation may be output to the user or stored, offering an evidence-based prediction of the impact of specific interventions on the subject's biomarker-linked condition. Specifically, this feature may enable proactive health management by allowing users to explore “what-if” scenarios, informing decision-making for personalized treatment plans or lifestyle modifications.

In some aspects, the foregoing steps may be performed in the listed order above. In other aspects, the foregoing steps may be performed in a different order than listed above. In some aspects, two or more steps listed above may be performed concurrently.

### Exemplary Embodiments

The concepts described herein may be applied specifically to the relative abundances of microbial taxa within the human gut microbiome, enabling a sophisticated analysis of health conditions based on microbiome data. Given the rapidly growing body of microbiome research, with thousands of studies published monthly, the system may harness this wealth of data to accurately characterize an individual's microbiome profile in the context of disease risk, health conditions, or potential treatment responses.

To determine the relevance and reliability of various microbiome findings, a quality score may be assigned to each study. The human microbiome's complexity, influenced by individual differences, diverse data acquisition methods, sequencing variations, and environmental factors, means that study reliability may vary significantly. Larger studies, or those published in high-impact journals, generally offer more reliable findings. The aspects described herein account for such variations by creating a quality score for each study based on parameters like study size and journal impact factor, allowing the system to weight each study accordingly in its calculations. Each study may contain multiple findings on the associations between specific microbial taxa and health conditions, which are integrated through a quality score model. The quality score for each finding may be represented as a vector, where each component may correspond to an organism's association with a health condition. This scoring system may help quantify both the study's evidence strength and its consistency with other relevant findings, using normalization and distance metrics to further refine accuracy.

The quality score of finding i may be defined by combining the strength of evidence—defined by the study size and impact factor—and consistency of the finding compared to other relevant findings. In an aspect, f_i may be the O-dimensional vector of an i-th finding for a condition, where O is the number of organisms.

$$\vec{{f}_{i}} = {\{ \begin{matrix}+ 1 & If overabundance of organism j is associated with condition \\ - 1 & If underabundance of organism j is associated with condition \\ 0 & Otherwise\end{matrix} \}}_{∀ j}$$

The j-th value of f_i indicates if and how the j-th organism is associated with a condition. The evidence strength of the i-th finding may further be defined as s_i,

$${s}_{i} = NORM ( Study Size ) + NORM ( Impact Factor )$$

Where NORM may correspond to a min-max normalization to the range [0,1] over all study sizes and impact factors present in the initial database. s_i may be redefined to include other variables, such as the number of citations and publication date. To illustrate how inconsistency of an i-th finding out of N findings may be defined, the central vector of this condition may further be defined as a weighted sum finding vectors, weighted by the evidence strength.

$$\vec{c} = \frac{1}{{\sum }_{i = 1}^{N} {s}_{i}} {\sum }_{i = 1}^{N} {s}_{i} \vec{{f}_{i}}$$

Inconsistency of the i-th finding may be defined as:

I(\vec{f}ᵢ)=cos(\vec{f}ᵢ;\vec{c})

where cos is a cosine distance function measuring how far apart a finding is from the central vector. The cosine distance may be replaced by other distance metrics (e.g., Manhattan distance, Euclidean distance, Chebyshev distance, etc.). This inconsistency may be transformed to penalize findings highly inconsistent from other papers. The final quality score of finding i may be written as:

$${q}_{i} = \frac{1}{I ( \vec{{f}_{i}} )}$$

and this equation may reflect both strength and consistency of the evidence. The final transformation q_i may be replaced with any function that increases as I(f_i) decreases and decreases as I(f_i) increases.

In an aspect, the system may utilize the GMrepo database as a reference point for microbiome data distributions across healthy populations. GMrepo is a well-curated, comprehensive dataset from public microbiome studies, uniformly processed under a consistent bioinformatics pipeline. GMrepo may provide empirical abundance distributions for microbial taxa, serving as a comparative baseline. Criteria for selecting GMrepo data include samples from healthy individuals aged five or older, though samples with unspecified ages are also included to maximize data volume. Poor-quality samples (e.g., those with biologically implausible abundance totals or single-organism dominance) are excluded, resulting in a refined dataset of 8,124 samples representing the healthy reference population. The GMrepo demographic profile further provides valuable contextual metadata, such as geographic distribution, gender, age, and BMI statistics, helping to delineate a diverse yet reliable baseline population.

### Calculating Associations Based on the Literature

This section details the method by which associations are computed between an input set of microbiome relative abundances and conditions of interest, using data from scientific literature. This approach may begin with recognizing that findings in the observational microbiome literature often report a statistically significant overabundance or underabundance of specific organisms when comparing a condition (e.g., a disease state) to a healthy control population. To leverage these findings, the system may calculate the probability that a given organism, A, observed at certain abundance level, r, in a sample, would be more abundant than what is typically observed in a healthy population. This probability may be derived from the empirical cumulative distribution function (CDF), denoted as:

P(r)=CDF_{A}(r)

Where CDF_{A} corresponds to the empirical cumulative distribution function of the organism A, found in the (GMrepo) dataset for healthy individuals. Similarly, the probability that an organism found in a sample at abundance level r is less abundant relative to a randomized healthy individual is:

$$P ( r ) = 1 - {CDF}_{A} ( r )$$

In an aspect, given a sample abundance vector \vec{r}, where i-th value is the abundance of the i-th organism, the association factor of the j-th finding between a condition and over or underabundance of organisms, may then be written as:

$${S}_{j} ( \vec{r} ) = {q}_{j} {\sum }_{i ∈ {O}_{j}} {CDF}_{i} ( {r}_{i} ) [ [ [ i is associated with overabundance + ( 1 - {CDF}_{i} ( {r}_{i} ) ] ] [ \text{} i is associated with underabundance ]$$

Where I is an indicator function resulting in 1 if the condition as indicated by the logical index in brackets is satisfied or 0, otherwise. O_j is the set of organisms that j-th finding found association with a condition and q_j is the quality score of the j-th finding.

In an aspect, the association factor for abundance level rand a condition may be determined by aggregating the association factor across N findings by using a median. The median may be replaced by other aggregating functions, such as average, weighted sum/median, etc.

$$\overline{S} = MEDIAN ( {S}_{j} ( \vec{r} ) \vert j ∈ \{ 1 , … , N \} )$$

To create a firm determination of association, the S value may be thresholded (e.g., at a threshold of 0.5), stratified (multiple thresholds) or output as a real value.

### Generating Actionable Insights

This section outlines how the system may evaluate different types of studies to provide targeted recommendations based on the analysis of microbiome data. This process may be categorized into three main types of actions, each reflecting how microbiome changes in response to interventions and how these changes may inform personalized recommendations.

In an aspect, a “Type 1” action paper may involve studies that measure the microbiome profiles of individuals with a disease before undergoing a specific action (e.g., a treatment). These studies may distinguish between responders and non-responders to assess the action's effectiveness. For instance, a study may explore the response to Infliximab in a population with Crohn's Disease by comparing microbiome profiles of those who benefited (responders) and those who did not (non-responders). If a new sample's microbiome profile aligns with that of known responders, the system may infer a likely positive response to the treatment. This type of action helps personalize treatment by matching the subject's microbiome to responder profiles, enabling predictions about the effectiveness of specific interventions.

In an aspect, a “Type 2” action may focus on the effect of an action on microbiomes in individuals without a known disease. Here, studies may compare microbiomes of healthy individuals who undertake an action (e.g., adopting a vegetarian diet) to those who do not (e.g., a control or placebo group). The system may analyze the microbiome data post-intervention to understand how the action modifies the microbial composition. This type of analysis provides insights into preventive measures or lifestyle changes that could benefit healthy individuals by indicating potential microbiome shifts associated with positive health outcomes.

In an aspect, a “Type 3” action paper may be similar to Type 2, but may specifically apply to individuals with a known condition. The analysis here may involve comparing diseased individuals who receive an action (e.g., a specific drug or therapeutic regimen) to those who receive a placebo or no intervention. For example, a study might compare the microbiome changes in inflammatory bowel disease (IBD) patients who are administered Infliximab versus those given a placebo. This type of action helps evaluate the effectiveness of actions within diseased cohorts, guiding treatment options by indicating whether a specific action is likely to induce beneficial microbiome alterations.

In terms of calculation, Type 1 action studies help predict how similar a subject's microbiome is to that of responders by assessing the relative abundances of organisms compared to a healthy baseline. The system may compute probabilities that specific organisms are more or less abundant in the subject compared to the healthy population using empirical cumulative distribution functions (CDFs). For instance, if we assume that the base population is healthy individuals and we assume that for a condition α and for organism A the relative abundance is r_{A}, then the probability that the relative abundance of A is more abundant than the healthy population may be:

P(r)=CDF_{A}(r)

and the probability that relative abundance of A is less abundant than the healthy population may be:

$$P ( r ) = 1 - {CDF}_{A} ( r )$$

In an aspect, the expression of the evidence in the literature that the action may be more effective may be written as:

$${S}_{j} ( \vec{r} ) = {q}_{j} {\sum }_{i ∈ {O}_{j}} {CDF}_{i} ( {r}_{i} ) [ [ [ i is associated with overabundance + ( 1 - {CDF}_{i} ( {r}_{i} ) ] ] [ \text{} i is associated with underabundance ]$$

Where I is an indicator function resulting in 1 if the condition as indicated by the logical index in brackets is satisfied or 0, otherwise. Oⱼ is the set of organisms that j-th finding found association with a condition and qⱼ is the quality score of the j-th finding. The findings may be aggregated and an association score calculated using median. The median may be replaced by other aggregating functions, such as average, weighted sum/median, etc.

$$\overline{S} = MEDIAN ( {S}_{j} ( \vec{r} ) \vert j ∈ \{ 1 , … , N \} )$$

In an aspect, another example of a Type 1 action may be to predict whether a person with diabetes (or obesity) may respond to a GLP-1 agonist. Assuming that the population was known to be diabetic (or obese), then the CDF of the diabetic (or obese) population may be utilized for a given relative abundance r.

For Type 2 actions, the focus may shift to evaluating how the microbiome profile changes upon the action's introduction. The system may compare the probability density functions (PDFs) of specific organisms pre- and post-action to identify potential alterations linked to positive outcomes. More particularly, assume for a condition α and for organism j the relative abundance is rⱼ. The probability density function associated with this organism is pdfⱼ(r). S_{α} is the association factor of a condition α. In an aspect, the association factor for the condition may be recalculated by considering the organism's abundance distributions and the quality scores of findings. For example, for each finding i and the weight of the finding qᵢ,
Adjusted relative abundance of organism j following the action for finding i is r_{i,j}*.
   If qᵢ>0, then the expected abundance r_{i,j}*=∫ᵣⱼ¹rpdfⱼ(r)dr
   qᵢ<0, then the expected abundance r_{i,j}*=∫₀^{rj}rpdfⱼ(r)dr
qᵢ=0, then r_{i,j}*=rⱼ
The adjusted abundance of organism j over all findings related to the action is

$${r}_{j}^{*} = \frac{{\sum }_{i} {q}_{i} {r}_{i , j}^{*}}{{\sum }_{i} {q}_{i}}$$

The adjusted abundance vector of this action becomes is \vec{r}*={rⱼ*}
The adjusted association score of the condition α after taking action is S_{α}(\vec{r}*)

In an aspect, Type 3 actions may extend the Type 2 analysis but use a diseased population as the reference group. This ensures that the association score reflects how the auction influences microbiome profiles relative to the initial diseased state, providing a more nuanced view of how treatments or interventions may alter health trajectories in affected populations.

### Food Groups

This section emphasizes the emerging importance of studying the impact of individual foods on the human gut microbiome and the potential to make dietary recommendations based on this research. Specific foods, such as fermented products like yogurt, kimchi, and kombucha, have been associated with positive changes in the gut microbiome, such as increased microbial diversity and reduced inflammatory markers. These changes are linked to various health benefits, prompting research into how different dietary choices can influence gut health. However, while there is growing interest in this area, the evidence base for the effects of individual foods is often limited, as studies on specific foods may be sparse or lack robust support.

To overcome the challenge of limited evidence for individual food items, the system may use an in-house food ontology to group foods into broader categories. This ontology connects specific food items to related food groups, enabling the aggregation of findings from studies that may focus on different but similar foods. For example, an apple may be classified as both a polyphenol-rich food and a high-fiber food. By categorizing the apple in these food groups, any studies linking polyphenol-rich or high-fiber foods to microbiome changes can be collectively considered when assessing the impact of eating apples. This approach strengthens the evidence by pooling data from various studies that may otherwise be considered separately, increasing the reliability of the system's dietary recommendations.

Using this food ontology, the system may merge findings related to slightly different but related actions or conditions, making it possible to calculate more comprehensive association scores or adjusted scores for specific foods and their effects on the microbiome. This technique may enhance the capability of the system to recommend dietary changes based on solid, aggregated evidence that considers the broader context of related foods and their collective impact. By integrating such nuanced and interconnected data, the system may offer personalized dietary suggestions that promote gut health and overall well-being, informed by a comprehensive understanding of food-microbiome interactions.

Refinement with New Data

In an aspect, this section describes how the system may enhance its predictive capabilities by incorporating new data to refine initial association scores. While the primary method combines multiple existing biomarker findings to determine an association score for a subject's condition or response, new data may be used to improve the accuracy and reliability of these predictions. In an aspect, the process may begin by calculating an initial association score for subjects based on the aggregated findings from the literature and other sources. This score provides a preliminary measure of the subject's association with specific conditions or outcomes.

To refine these initial scores, the system may evaluate the accuracy of its predictions by comparing them with known outcomes. This may involve determining a measure of error, which may be computed in various ways. One approach is to threshold the initial association scores and compare them against actual diagnostic results for each subject. Another method may involve assessing the system's predicted efficacy of an action, such as a drug treatment, and comparing it with the actual measured outcome, like changes in weight or BMI for subjects taking a GLP-1 agonist. This comparison helps identify discrepancies between the system's predictions and real-world results.

With this error data, the system may employ machine learning techniques to train a model that predicts the measure of error for each subject's initial association score. A variety of machine learning models may be applied, including neural networks, residual networks, support vector machines, random forests, and other deep learning methods. The trained model effectively learns a correction factor that may be used to adjust future association scores. Once trained, the machine learning system may predict the error for new subjects with new biomarker data. The revised association score is then determined by combining the initial association score and the predicted measure of error. This combination may be performed by subtracting the predicted error from the initial score or using other suitable mathematical methods. In this way, the machine learning technique refines the association score, resulting in a more accurate and reliable assessment for new data inputs. This iterative approach allows the system to evolve and improve its predictive accuracy over time as more data becomes available, ensuring that the analyses remain robust and reflective of real-world outcomes.

In general, any process discussed in this disclosure that is understood to be computer-implementable, such as the processes illustrated in FIG. 2, may be performed by one or more processors of a computer system, such as computer system 100 described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as computer system 100, may include one or more computing devices. If the one or more processors of the computer systems are implemented as a plurality of processors, the plurality of processors may be included in a single computing device or distributed among a plurality of computing devices. If the computer system 100 comprises a plurality of computing devices, the memory of the computer system 100 may include the respective memory of each computing device of the plurality of computing devices.

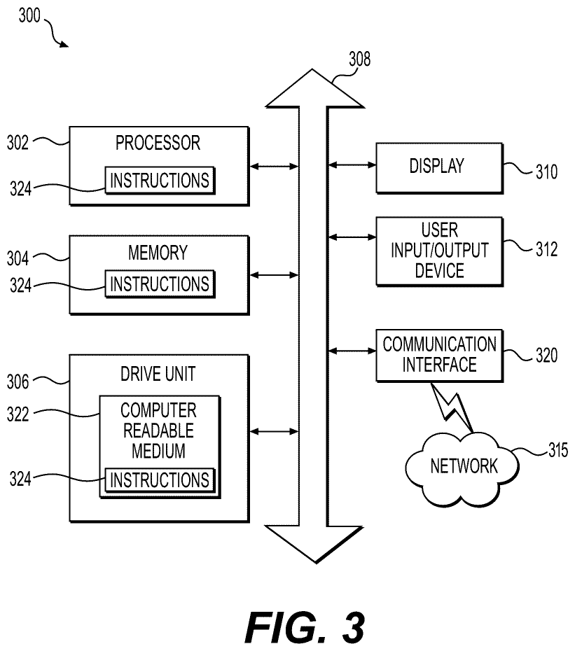

FIG. 3 is a simplified functional block diagram of a computer system 300 that may be configured as a computing device for executing the process illustrated in FIG. 2, according to exemplary embodiments of the present disclosure. FIG. 3 is a simplified functional block diagram of a computer that may be configured as the computer system 100 according to exemplary embodiments of the present disclosure. In various embodiments, any of the systems herein may be an assembly of hardware including, for example, a data communication interface 320 for packet data communication. The platform also may include a central processing unit (“CPU”) or processor 302, in the form of one or more processors, for executing program instructions. The platform may include an internal communication bus 408, and a storage unit 306 (such as ROM, HDD, SDD, etc.) that may store data on a computer readable medium 322, although the system 300 may receive programming and data via network communications via electronic network 325 (e.g., voice, video, audio, images, or any other data over the electronic network 325). The system 300 may also have a memory 304 (such as RAM) storing instructions 324 for executing techniques presented herein, although the instructions 324 may be stored temporarily or permanently within other modules of system 300 (e.g., processor 302 and/or computer readable medium 322). The system 300 also may include input and output devices 312 and/or a display 310 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

In general, any process discussed in this disclosure that is understood to be performable by a computer may be performed by one or more processors. Such processes include, but are not limited to: the process shown in FIG. 2, and the associated language of the specification. The one or more processors may be configured to perform such processes by having access to instructions (computer-readable code) that, when executed by the one or more processors, cause the one or more processors to perform the processes. The one or more processors may be part of a computer system (e.g., one of the computer systems discussed above) that further includes a memory storing the instructions. The instructions also may be stored on a non-transitory computer-readable medium. The non-transitory computer-readable medium may be separate from any processor. Examples of non-transitory computer-readable media include solid-state memories, optical media, and magnetic media.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for determining an association between a subject and at least one condition, the computer-implemented method comprising:
receiving, at a computing device associated with a system, population biomarker data from a plurality of systems that include study data for a plurality of studies;
receiving, at the computing device, one or more population probability values;
receiving, at the computing device, one or more biomarker levels associated with the subject;
analyzing, using a processor associated with the system, the one or more biomarker levels against the population biomarker data and the one or more population probability values;
generating, based on the analyzing and using the processor, an association score between the one or more biomarker levels and the at least one condition; and
outputting, using the processor, the association score to a display of the computing device.

2. The computer-implemented method of claim 1, wherein the plurality of studies comprise a study type including at least one of: a peer-reviewed journal, a preprint, or a report.

3. The computer-implemented method of claim 1, further comprising:
analyzing, using the processor, one or more factors associated with the study data from each of the plurality of studies;
determining, based on the analyzing and using the processor, a quality score associated with each of the plurality of studies; and
excluding, prior to analyzing the one or more biomarker levels and using the processor, the population biomarker data associated with each of the plurality of studies having the quality score under a predetermined threshold.

4. The computer-implemented method of claim 3, wherein the one or more factors include one or more of: a sample size, a journal impact factor, a citation count, and a publication date.

5. The computer-implemented method of claim 1, wherein the generating the association score comprises:
calculating, using the processor, for each of the plurality of studies, a likelihood that the one or more biomarker levels associated with the subject would appear in the population biomarker data;
calculating, using the processor, a second association score between the subject and one or more populations associated with the population biomarker data;
identifying, using the processor, a target population of the one or more populations having a largest second association score; and
generating, using the processor, the association score between the one or more biomarker levels and the at least one condition based on a reference condition associated with the target population.

6. The computer-implemented method of claim 1, wherein the generating the association score comprises:
determining, using the processor, for each of the plurality of studies, a plurality of second association scores that each reflect a relationship between the one or more biomarker levels and one or more populations associated with the population biomarker data; and
combining, using the processor, the plurality of second association scores to generate the association score using a statistical method.

7. The computer-implemented method of claim 1, wherein the outputting comprises transmitting the association score to a designated storage location.

8. The computer-implemented method of claim 1, further comprising:
receiving, using the processor, a proposed action for affecting the one or more biomarker levels;
receiving, using the processor, study data for one or more studies that compare one or more biomarkers between a first population that has taken the proposed action and a second population that has not taken the proposed action;
determining, based on the study data and using the processor, an impact of the proposed action; and
outputting, using the processor, the impact to a display of the computing device.

9. The computer-implemented method of claim 9, further comprising:
recalculating, using the processor, the association score with a hypothesized biomarker profile representing the impact of the proposed action.

10. A system for determining an association between a subject and at least one condition, the system comprising:
one or more processors; and
one or more computer readable media storing instructions that are executable by the one or more processors to perform operations comprising:
receiving population biomarker data from a plurality of systems that include study data for a plurality of studies;
receiving one or more population probability values;
receiving one or more biomarker levels associated with the subject;
analyzing the one or more biomarker levels against the population biomarker data and the one or more population probability values;
generating, based on the analyzing and using the processor, an association score between the one or more biomarker levels and the at least one condition; and
outputting, using the processor, the association score to a display of the computing device.

11. The system of claim 10, wherein the plurality of studies comprise a study type including at least one of: a peer-reviewed journal, a preprint, or a report.

12. The system of claim 10, further comprising:
analyzing one or more factors associated with the study data from each of the plurality of studies;
determining, based on the analyzing, a quality score associated with each of the plurality of studies; and
excluding, prior to analyzing the one or more biomarker levels and using the processor, the population biomarker data associated with each of the plurality of studies having the quality score under a predetermined threshold.

13. The system of claim 13, wherein the one or more factors include one or more of: a sample size, a journal impact factor, a citation count, and a publication date.

14. The system of claim 11, wherein the generating the association score comprises:
calculating, using the processor, for each of the plurality of studies, a likelihood that the one or more biomarker levels associated with the subject would appear in the population biomarker data;
calculating, using the processor, a second association score between the subject and one or more populations associated with the population biomarker data;
identifying, using the processor, a target population of the one or more populations having a largest second association score; and
generating, using the processor, the association score between the one or more biomarker levels and the at least one condition based on a reference condition associated with the target population.

15. The system of claim 11, wherein the generating the association score comprises:
determining, for each of the plurality of studies, a plurality of second association scores that each reflect a relationship between the one or more biomarker levels and one or more populations associated with the population biomarker data; and
combining the plurality of second association scores to generate the association score using a statistical method.

16. The system of claim 11, wherein the outputting comprises transmitting the association score to a designated storage location.

17. The system of claim 11, further comprising:
receiving a proposed action for affecting the one or more biomarker levels;
receiving study data for one or more studies that compare one or more biomarkers between a first population that has taken the proposed action and a second population that has not taken the proposed action;
determining, based on the study data, an impact of the proposed action; and
outputting the impact to a display of the computing device.

18. The system of claim 17, further comprising:
recalculating the association score with a hypothesized biomarker profile representing the impact of the proposed action.

19. A non-transitory computer-readable medium storing computer-executable instructions which, when executed by a system, cause the system to perform operations comprising:
receiving, at a computing device associated with a system, population biomarker data from a plurality of systems that include study data for a plurality of studies;
receiving, at the computing device, one or more population probability values;
receiving, at the computing device, one or more biomarker levels associated with the subject;
analyzing, using a processor associated with the system, the one or more biomarker levels against the population biomarker data and the one or more population probability values;
generating, based on the analyzing and using the processor, an association score between the one or more biomarker levels and the at least one condition; and
outputting, using the processor, the association score to a display of the computing device.

20. The non-transitory computer-readable medium of claim 19, wherein the plurality of studies comprise a study type including at least one of: a peer-reviewed journal, a preprint, or a report.
